(19) United States
(12) Patent Application Publication
(10) Pub. No.: US 2022/0054782 A1
(43) Pub. Date: Feb. 24, 2022

(54) SPEAKING VALVE PROTECTOR

(71) Applicant: KAPITEX HEALTHCARE LIMITED, Yorkshire (GB)

(72) Inventors: Ian David WORTHINGTON, Yorkshire (GB); Alexander James BRIERLEY, Yorkshire (GB)

(21) Appl. No.: 17/298,759

(22) PCT Filed: Feb. 14, 2020
(86) PCT No.: PCT/EP2020/053958

§ 371 Date: Jun. 1, 2021

(30) Foreign Application Priority Data
Feb. 14, 2019 (GB) 1902066.8

(51) Int. Cl. A61M 16/04; A61F 2/20

(52) U.S. Cl. CPC A61M 16/0468; A61F 2002/206; A61F 2/20

(57) ABSTRACT

A shower shield for use with a tracheostoma, having a chamber with a port for connection to a speaking valve or tracheostomy base plate, and a downwardly-facing aperture, and further provided with a resiliently-deformable wall region so that a user's finger pressure on the wall region said region can actuate the speaking valve or occlude a tracheostoma.

### FIELD OF THE INVENTION

The invention relates to protective shields used to prevent, or decrease, ingress of water droplets into the trachea of a user having a tracheostomy.

### BACKGROUND AND PRIOR ART

Speaking valves are known in themselves, and are used to control, or assist the control of the flow of air through a tracheostoma—a passage formed between the trachea of a human subject, and the outside air.

One particular, and typical use, will be described with reference to FIGS. 1 and 2. FIG. 1 shows a schematic cross-section through a human subject 1 who has undergone a laryngectomy. FIGS. 2A and 2B illustrate an enlargement of the region enclosed by the dashed-line circle. The removal of the larynx is often a consequence of throat cancer. Illustrated are the trachea 2, or windpipe, connecting the lungs to the tracheostoma 3, and also the oesophagus 4 connecting the stomach to the mouth 5. In order to allow a person to speak, in the absence of a larynx, a voice prosthesis 6 is fitted within a fistula made between the trachea 2 and the oesophagus 6. The voice prosthesis acts as a one-way valve, preventing food and drink within the oesophagus 6 reaching the lungs via the trachea 2. A speaking valve 7 is provided within the tracheostoma, between the outside air and the opening to the trachea 2. In its non-speaking configuration, the speaking valve 7 allows the flow of air into and out of the trachea as illustrated by the arrow in FIG. 2A, thereby allowing the individual to breathe.

When the individual wishes to speak, they take air into their lungs with the speaking valve 7 in the configuration shown in FIG. 2A, and then cause the valve 7 to close while exhaling the air. With the speaking valve 7 closed, air is forced through the voice prosthesis 6 and into the upper region of the oesophagus as indicated by the arrow in FIG. 2B. This flow of air vibrates soft tissue within the patient's oesophagus causing sound to be produced that the individual can control to produce speech.

In its simplest form, a speaking valve 7 could constitute an aperture through the tracheostoma that the individual can close by covering the aperture with a finger or thumb. In more advanced valves, a valve element occluding the aperture might be provided, biased into an open position. An individual can then push and hold the valve element into a closed position while speaking.

One difficulty for users with this kind of intervention is that there is a risk of water entering the tracheostoma and getting into the user's lungs, as the user has no mechanism to close the trachea. If the tracheostoma becomes immersed in water, there is a real risk of the user drowning. As a result, it is usually recommended that tracheostomy users do not swim, at least without specialised equipment and very careful precautions.

However, on a daily basis, tracheostomy users also have deal with everyday activities such as showering and even walking in heavy rain. Showering can pose a risk of water entering the trachea, although it is possible for users to reduce the risk by facing away from the shower jets when showering.

Alternatively, and much safer for the user, a shower shield may be used. FIG. 3 illustrates a known shower shield, generally indicated by 8, in perspective view. FIG. 4 shows the same shield in cross-sectional view. The shield 8 has the form of a chamber 9 having a port 10 to which a tracheostomy speaking valve may be fitted, and an aperture 11 that may be positioned to face downwards, in use. The chamber typically has a flat side 12 opposite the port, to minimise the bulk of the device.

FIG. 5 illustrates a known speaking valve 13 having a moveably-mounted occluder plate 14 that may be positioned by a user's finger pressure to prevent or resist air-flow through the speaking valve 13. Such valves are known in the art, and are described in e.g WO03061531 and EP2367588. The occluder plate 14 is typically biased towards the open position by means of e.g. resilient foam 15 or a spring 16, as illustrated in FIG. 6. The end of the speaking valve opposite the occluder plate 14 is provided with a connector 17 to allow the valve 13 to connected to a base plate (not illustrated), typically adhesively fixed to user's skin around the tracheostoma.

At the end of the valve closest to the occluder plate 14 the valve 13 is provided with a connector 18 to allow the valve 13 to be connecter to a corresponding connector 19 on the shield 8.

In this way, the shield may be oriented with its air inlet aperture 11 pointing downwards, thereby preventing downwardly falling water droplets (e.g. from a shower) from entering the user's trachea.

While this type of shield works well in preventing such water entry, it blocks access to the occluder plate 14 of a speaking valve (or, equivalently, an aperture leading to the user's trachea) thereby preventing the user from speaking while using the device. This can be important because it prevents a user from talking, or perhaps calling for assistance, while in the shower, or walking in a heavy rainstorm. This is at least inconvenient, if not dangerous, and could also lead to a user not using the shield when it would be advisable to do so.

It is amongst the objects of the invention to attempt a solution to this and other problems,

### SUMMARY OF THE INVENTION

Accordingly, the invention provides a shower shield for use with a tracheostoma, said shield comprising a chamber having: (a) a port in communication with the interior of said chamber for connection to a tracheostomy speaking valve or tracheostomy base plate; and (b) an aperture that may be positioned to face downwards, in use; wherein the wall of said chamber opposite said port is provided with a resiliently-deformable region so that a user's finger pressure on said region can cause said region to deform and at least contact a speaking valve or base plate when attached to said port.

Preferably, said resiliently-deformable region comprises an elastomeric membrane, and more preferably said elastomeric membrane is co-moulded with a plastics chamber.

In any aspect of the invention, it is also preferred that the interior surface of said resiliently-deformable region is provided with a protuberance extending towards said port. More preferably, said protuberance extends so as to touch the surface of a speaking valve when attached to said port and said resiliently-deformable region is in its undeformed configuration.

### BRIEF DESCRIPTION OF THE FIGURES

The invention will be describer with reference to the accompanying drawings, in which:

FIGS. 1 and 2 illustrate the context for use of a speaking valve protector of the invention with a cross-section of the head of a user;

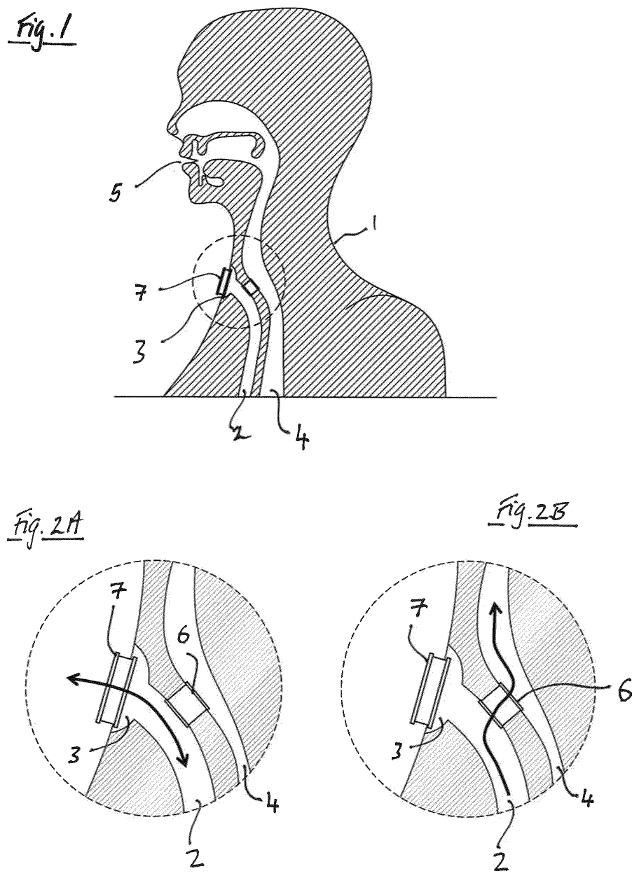

FIGS. 3-4 illustrate in perspective and cross-sectional views respectively a known shower shield;

FIGS. 5-6 illustrate, in cross-sectional views, known embodiments of speaking valves; and

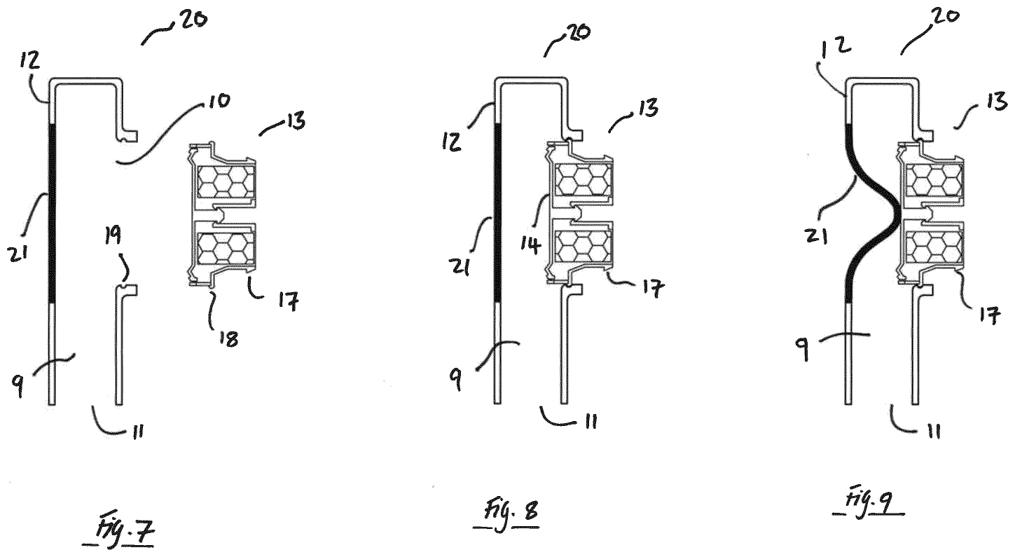

FIGS. 7-12 illustrate, in cross-sectional views, embodiments of shower shields of the invention, in use with speaking valves.

### DESCRIPTION OF PREFERRED EMBODIMENTS

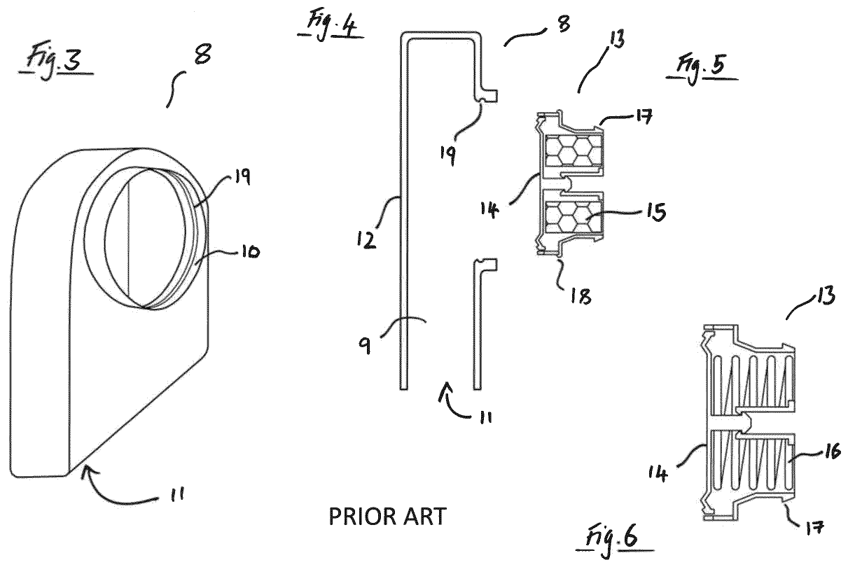

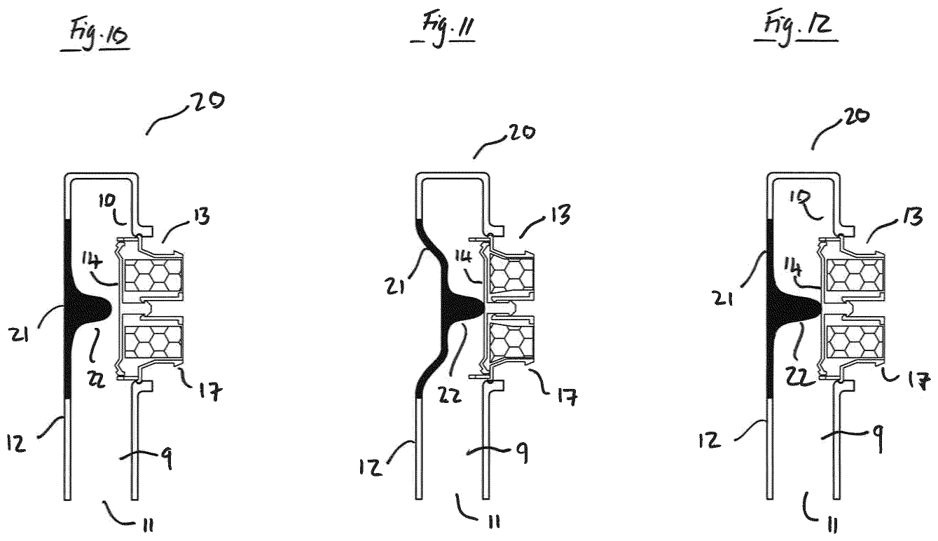

FIGS. 7-9 illustrate, in cross-sectional view, an embodiment of a shower shield of the invention, generally indicated by 20, together with a speaking valve 13. The perspective view is essentially the same as FIG. 3.

The shower shield 20 comprises a chamber 9 having a port 10 to which a tracheostomy speaking valve 13 may be fitted, and an aperture 11 that may be positioned to face downwards, in use. In this embodiment, a connector 19 is provided on the port 10 to connect the shield 20 to a corresponding connector 18 on a speaking valve to which the shower shield 20 is to be connected. In this embodiment, the connectors 18, 19 are illustrated as an interacting circumferential indent and detent, but other configurations are possible, and not crucial to the operation of the device, other than the connector should be water resistant and substantially air resistant.

The shower shield 20 may be connected to a speaking valve 13 as illustrated in FIG. 8.

On the forward-facing face 12 of the shower shield there is provided a resiliently-deformable region 21. The region 21 is located opposite the position of the port 10. The resiliently-deformable region 21 is configured such that finger pressure from a user can deform the region 21 to an extent that it can at least touch the speaking valve 13, as illustrated in FIG. 9.

Further pressure on the region 21 would serve to actuate the speaking valve in a known fashion if the valve were provided with e.g. a movable occluder plate as described above. Alternatively, the speaking valve might comprise a cap with a hole in it, and moving the deformable region 21 to the position illustrated in FIG. 9 would serve to allow vocalization.

The resiliently-deformable region 21 may comprise an elastomeric membrane. This could, e.g. be co-moulded with the rest of the shower shield using an elastomeric plastics for the resiliently-deformable region 21 and an essentially rigid plastics for the rest of the shield. Alternatively, an essentially rigid plastics shield could be provided having a hole where the resiliently-deformable region is desired, and a resiliently-deformable elastomeric cover placed over the shield (avoiding the port 10 and the aperture 11) to provide the required deformable region 21. In other embodiments, an essentially rigid plastics shield could again be provided having a hole where the resiliently-deformable region is desired, and a plug formed of resiliently-deformable material could then be disposed in the hole to produce the desired structure.

FIGS. 10-11 illustrate, in cross-sectional view, a further embodiment of a shower shield of the invention, generally indicated by 20. Features in common with the embodiment illustrated in FIGS. 7-10 are numbered correspondingly.

In this embodiment, the resiliently-deformable region 21 is provided with an inwardly-extending protuberance 22 on its inside surface, the protuberance extending towards the port 10. The protuberance 22 could form part of the deformable region, or could alternatively be attached to the inside surface of a separate deformable region. By the addition of this feature, the region 21 does not have to be deformed as much as in the embodiment illustrated in FIGS. 7-10 in order for the protuberance 22 to touch and then actuate the speaking valve, as illustrated in FIG. 11 where the speaking valve is shown in its closed position.

FIG. 12 illustrates a yet further development of the shower shield illustrated in FIGS. 10-11. Again, features in common with previously described embodiments are numbered correspondingly.

In this embodiment, the protuberance is so sized as to touch the surface of the occluder plate 14 of the speaking valve 13 when the region 21 is in its undeformed state, as illustrated. In this way, the movement required to be imparted by the user onto the resiliently-deformable region 21 is the same as when they use the speaking valve 13 without the use of a shower shield.

1. A shower shield for use with a tracheostoma, said shield comprising a chamber having:
(a) a port in communication with the interior of said chamber for connection to a tracheostomy speaking valve or tracheostomy base plate; and
(b) an aperture that may be positioned to face downwards, in use;
wherein the wall of said chamber opposite said port is provided with a resiliently-deformable region so that a user's finger pressure on said region can cause said region to deform and at least contact a speaking valve or base plate when attached to said port.

2. A shower shield according to claim 1 in which said resiliently-deformable region comprises an elastomeric membrane.

3. A shower shield according to claim 3 wherein said elastomeric membrane is co-moulded with a plastics chamber.

4. A shower shield according to claim 1 wherein the interior surface of said resiliently-deformable region is provided with a protuberance extending towards said port.

5. A shower shield according to claim 4 wherein said protuberance extends so as to touch the surface of a speaking valve when attached to said port and said resiliently-deformable region is in its undeformed configuration.
